(19) United States
(12) Patent Application Publication
(10) Pub. No.: US 2025/0255236 A1
(43) Pub. Date: Aug. 14, 2025

(54) BROCCOLI VARIETY 25-BO239 RZ

(71) Applicant: RIJK ZWAAN ZAADTEELT EN ZAADHANDEL B.V., De Lier (NL)

(72) Inventors: Carlos Gil-Albert Armero, De Lier (NL); Laura Balenzategui Iparraguirre, De Lier (NL); José Antonio Hernández Torres, De Lier (NL)

(21) Appl. No.: 19/052,708

(22) Filed: Feb. 13, 2025

Related U.S. Application Data
(60) Provisional application No. 63/553,440, filed on Feb. 14, 2024.

(51) Int. Cl. A01H 6/20; A01H 5/12

(52) U.S. Cl. CPC A01H 6/203; A01H 5/12

(57) ABSTRACT

The present invention relates to a Brassica oleracea var. italica seed designated 25-BO239 RZ. The present invention also relates to a Brassica oleracea var. italica plant produced by growing the 25-BO239 RZ seed. The invention further relates to methods for producing the broccoli cultivar, represented by broccoli variety 25-BO239 RZ.

### RELATED APPLICATIONS AND INCORPORATION BY REFERENCE

Priority is claimed to U.S. Provisional Application Ser. No. 63/553,440, filed Feb. 14, 2024, which is incorporated herein by reference in its entirety.

All documents cited or referenced herein (“herein cited documents”), and all documents cited or referenced in herein cited documents, together with any manufacturer's instructions, descriptions, product specifications, and product sheets for any products mentioned herein or in any document incorporated herein by reference, are hereby incorporated herein by reference, and can be employed in the practice of the invention.

### FIELD OF THE INVENTION

The present invention relates to a new type of broccoli (Brassica oleracea var. italica), including variety designated 25-BO239 RZ, this new type of broccoli having a set of characteristics including but not limited to a plant of the variety having a short to very short plant height and a head size of about 2 to about 10 cm, preferably about or greater than 2 cm to less than about 10 cm, when directly sown in high density ranging from 20 to 100 plants per m², e.g., each whole integer between 20 and 100 (including 20 and 100) plants per m² such as 20, or 21, or 22, or 23, or 24, or 25, or 26, or 27, or 28, or 29, or 30, or 31, or 32, or 33, etc. to 100 plants per m².

### BACKGROUND OF THE INVENTION

Broccoli is an edible vegetable plant that belongs to the highly polymorphic species Brassica oleracea and is classified in the Italica cultivar group. Like cauliflower, a broccoli plant produces a large edible flowerhead called a curd. However, unlike cauliflower the curd contains clearly distinguishable flower buds. The two most common types of broccoli are Calabrese or heading broccoli and sprouting broccoli. Calabrese type broccoli plants usually form a single green head having a diameter of approximately 10 to 20 cm. Sprouting broccoli produces many smaller heads on different shoots.

Broccoli is considered a cool weather crop. Cultivation best takes place with an average day temperature between 18° C. and 23° C. Broccoli can be sown directly, or first sown indoors before being transplanted to the field. Planting density for regular broccoli cultivation ranges from 30,000 to 100,000 plants per hectare.

Harvesting of broccoli heads is predominantly done manually. There have been attempts to mechanically harvest broccoli heads, but the plant architecture of a regular broccoli makes mechanical harvesting difficult because leaves surrounding the head can jam regular harvest equipment.

The head of a broccoli plant is usually sold as fresh produce. However, like with other vegetables, consumers are more and more demanding convenient solutions such as ready-to-eat and individualized portioning. For broccoli this means that the broccoli head is cut up in individualized florets. This processing of the curd is labor intensive, produces a lot of waste and therefore is expensive. For example, florets of normal broccoli heads show yellow edges where the sunlight has not reached the plants. This is usually the part of the floret that was touching another floret during cultivation. These yellow edged are removed from the floret, because consumers find these unattractive.

### SUMMARY OF THE INVENTION

The present invention addresses the need expressed by relevant stakeholders by providing a new type of broccoli variety, that is suitable for mechanical harvest and has characteristics that lead to more efficient processing.

The present invention provides a new type of broccoli (Brassica oleracea var. italica) plant, including the variety designated 25-BO239 RZ, which exhibits a set of characteristics including but not limited to a short to very short plant height (with “short to very short plant height” defined herein with reference to the UPOV standard) and a head size of about 2 to about 10 cm when directly sown in high density ranging from 20 to 100 plants per m², preferably about or greater than 2 cm to less than about 10 cm, when directly sown in high density ranging from 20 to 100 plants per m², e.g., each whole integer between 20 and 100 (including 20 and 100) plants per m² such as 20, or 21, or 22, or 23, or 24, or 25, or 26, or 27, or 28, or 29, or 30, or 31, or 32, or 33, etc. to 100 plants per m². Representative seed comprising, or providing, and having the heritable genetic information for this combination of characteristics (including a short to very short plant height and a head size of about 2 to about 10 cm, preferably about or greater than 2 cm to less than about 10 cm (such as illustrated in the Figures and described herein, see, e.g., Brief Description of Drawings), when directly sown in high density ranging from 20 to 100 plants per m² (and including each whole integer between 20 and 100 and the endpoints of 20 and 100 plants per m²), have been deposited with the National Collections of Industrial, Marine and Food Bacteria (NCIMB) in Wellheads Place, Dyce, Aberdeen AB21 7 GB, UK and have been assigned NCIMB Accession No. 44350. This plant is herein referred to as the “broccoli plant of the invention” or “plant of the invention.”

The plant of the invention further exhibits the characteristics of extra short leaves as defined herein.

In one embodiment the plant of the invention has an average leaf length less than 30 cm, preferably less than 28 cm, most preferably less than 27.5 cm when sown in high density ranging from 20 to 100 plants per m², wherein the average leaf length is determined on at least 20 plants of which per plant all leaves are measured.

In one embodiment the plant of the invention is a plant of a broccoli variety designated 25-BO239 RZ. A sample of seeds of said broccoli variety, have been deposited with the National Collections of Industrial, Marine and Food Bacteria (NCIMB) in Wellheads Place, Dyce, Aberdeen AB21 7 GB, UK and have been assigned NCIMB Accession No. 44350.

In one embodiment, the invention provides a broccoli plant grown from the seed of broccoli (Brassica oleracea var. italica) variety 25-BO239 RZ.

In another embodiment, the invention provides a broccoli plant designated 25-BO239 RZ, representative seed of which having been deposited under NCIMB Accession No. 44350. An embodiment of such a broccoli plant can be a plant grown from seed having been deposited under NCIMB Accession No. 44350.

In one embodiment, the invention provides for a broccoli plant which comprises genetic information for exhibiting the aforementioned combination of characteristics (including a short to very short plant height and a head size of about 2 to about 10 cm, preferably about or greater than 2 cm to less than about 10 cm (such as illustrated in the Figures and described herein, see, e.g., Brief Description of Drawings), when directly sown in high density ranging from 20 to 100 plants per m² when directly sown in high density ranging from 20 to 100 plants per m² (and including each whole integer between 20 and 100 and the endpoints of 20 and 100 plants per m²), or for exhibiting all of the physiological and morphological characteristics of a plant of the invention (e.g., a plant having all of the physiological and morphological characteristics of a plant of the invention can be a plant grown from deposited seed), wherein the genetic information is as contained in a plant, a sample of seed of said variety having been deposited under NCIMB Accession No. 44350. The invention provides a broccoli plant of variety 25-BO239 RZ exhibiting the aforementioned combination of characteristics (including a short to very short plant and a head size of about 2 to about 10 cm, preferably about or greater than 2 cm to less than about 10 cm (such as illustrated in the Figures and described herein, see, e.g., Brief Description of Drawings), when directly sown in high density ranging from 20 to 100 plants per m² when directly sown in high density ranging from 20 to 100 plants per m² (and including each whole integer between 20 and 100 and the endpoints of 20 and 100 plants per m²), representative seed of which having been deposited under NCIMB Accession No. 44350, or representative seed of which comprising genetic information for expression by the plant exhibiting the aforementioned combination of characteristics having been deposited under NCIMB Accession No. 44350.

In one embodiment, the invention provides for a broccoli plant exhibiting the aforementioned combination of characteristics (including a short to very short plant height and a head size of about 2 to about 10 cm, preferably about or greater than 2 cm to less than about 10 cm (such as illustrated in the Figures and described herein, see, e.g., Brief Description of Drawings), when directly sown in high density ranging from 20 to 100 plants per m² when directly sown in high density ranging from 20 to 100 plants per m² (and including each whole integer between 20 and 100 and the endpoints of 20 and 100 plants per m²), or for exhibiting all the physiological and morphological characteristics of a plant of the invention, and having the genetic information for so exhibiting the combination of characteristics, or all the physiological and morphological characteristics of a plant of the invention (e.g., a plant having all of the physiological and morphological characteristics of a plant of the invention can be a plant grown from deposited seed), wherein the genetic information is as contained in a plant, a sample of seed of said variety having been deposited under NCIMB Accession No. 44350.

In an embodiment of the present invention, there also is provided a broccoli plant or parts of a broccoli plant of the invention, which can include parts of the broccoli plant exhibiting the aforementioned combination of characteristics (including a short to very short plant height and a head size of about 2 to about 10 cm, preferably about or greater than 2 cm to less than about 10 cm (such as illustrated in the Figures and described herein, see, e.g., Brief Description of Drawings), when directly sown in high density ranging from 20 to 100 plants per m² when directly sown in high density ranging from 20 to 100 plants per m² (and including each whole integer between 20 and 100 and the endpoints of 20 and 100 plants per m²), or exhibiting all the physiological and morphological characteristics of a plant of the invention (e.g., a plant exhibiting all of the physiological and morphological characteristics of a plant of the invention can be a plant grown from deposited seed), or parts of a broccoli plant having any or all of the aforementioned characteristics including one or more or all morphological and physiological characteristics tabulated herein, including parts of broccoli variety 25-BO239 RZ, wherein the plant parts are involved in sexual reproduction, which include, without limitation, microspores, ovaries, ovules, embryo sacs or egg cells and/or wherein the plant parts are suitable for vegetative reproduction, which include, without limitation, cuttings, roots, stems, cells or protoplasts and/or wherein the plant parts are tissue culture of regenerable cells in which the cells or protoplasts of the tissue culture are derived from a tissue such as, for example and without limitation, leaves, embryos, cotyledon, hypocotyls, meristematic cells, roots, root tips, flowers, seeds or stems. The plants of the invention from which such parts can come from include those wherein a sample of seed of which having been deposited under NCIMB Accession No. 44350 or broccoli variety designated 25-BO239 RZ, as well as seed from such a plant, plant parts of such a plant (such as those mentioned herein) and plants from such seed and/or progeny of such a plant, advantageously progeny exhibiting such combination of such traits, each of which, is within the scope of the invention; and such combination of traits.

In a further embodiment there is a plant regenerated from the above-described plant parts or regenerated from the above-described tissue culture. Advantageously such a plant has a set of characteristics including but not limited to a short to very short plant height and a head size of about 2 to about 10 cm when directly sown in high density ranging from 20 to 100 plants per m², or all morphological and/or physiological characteristics of broccoli variety 25-BO239 RZ and/or of a plant grown from seed, a sample of seed of which having been deposited under NCIMB Accession No. 44350-including without limitation such plants having all of the physiological and morphological characteristics of broccoli variety 25-BO239 RZ and/or of a plant grown from seed, a sample of seed of which having been deposited under NCIMB Accession No. 44350.

Accordingly, in still a further embodiment, there is provided a broccoli plant having all of the physiological and morphological characteristics of broccoli variety 25-BO239 RZ, a sample of seed of which having been deposited under NCIMB Accession No. 44350. Such a plant can be grown from the seeds, regenerated from the above-described plant parts, or regenerated from the above-described tissue culture. A broccoli plant having any of the aforementioned morphological or physiological characteristics recited herein, and a broccoli plant advantageously having all of the aforementioned characteristics recited herein, are preferred. Parts of such plants—such as those plant parts above-mentioned—are encompassed by the invention.

In a further aspect, the invention provides a method of vegetatively propagating a plant of the invention comprising (a) collecting tissue capable of being propagated from a broccoli plant of broccoli plant of the invention, a sample of seed of which having been deposited under NCIMB Accession No. 44350 and (b) cultivating the tissue to obtain proliferated shoots and rooting the proliferated shoots to obtain rooted plantlets. Optionally the invention further comprises growing plants from the rooted plantlets. Plantlets and plants produced by these methods, are encompassed by the invention.

In one embodiment, there is provided a method for producing a progeny of a broccoli plant of the invention, which can comprise crossing a plant of the invention with another broccoli plant, harvesting the resultant seed, and growing said seed.

In a further embodiment, a progeny plant is provided which is produced by this method, wherein said progeny exhibits a set of characteristics as discussed herein including but not limited to a short to very short plant height, and a head size of about 2 to about 10 cm when directly sown in high density ranging from 20 to 100 plants per m².

In another embodiment, a progeny plant is provided which is produced by the above method, wherein said progeny exhibits all the morphological and physiological characteristics of the broccoli variety designated 25-BO239 RZ, a sample of seed of said variety having been deposited under NCIMB Accession No. 44350.

Progeny of the plant of the invention can be modified in one or more other characteristics, in which the modification is a result of, for example and without limitation, mutagenesis or transformation with a transgene.

In still another embodiment, the present invention provides progeny of a plant of the invention produced by sexual or vegetative reproduction, grown from seed, regenerated from the above-described plant parts, or regenerated from the above-described tissue culture of the broccoli plant of the invention or a progeny plant thereof.

The invention further relates to a method for producing a seed of a plant derived from a broccoli plant of the invention comprising the steps of (a) crossing a plant of the invention, a sample of seed of which having been deposited under NCIMB Accession No. 44350, with a second broccoli plant, and (b) whereby seed of a plant derived from broccoli plant of the invention forms. This method can further comprise (c) crossing a plant grown seed of a broccoli plant derived a plant of the invention with a second broccoli plant to yield additional derived broccoli seed, (d) growing the additional derived broccoli seed of step (c) to yield additional derived broccoli plants, and (e) repeating the crossing and growing of steps (c) and (d) for an additional 3-10 generations to generate further derived broccoli plants, and (f) whereby seed of a derived broccoli plant forms. A seed produced by this method and a plant grown from said seed also form part of the invention.

Mutations can be introduced randomly by means of one or more chemical compounds, such as ethyl methane sulphonate (EMS), nitrosomethylurea, hydroxylamine, proflavine, N-methyl-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron exposure, X-rays, gamma irradiation, and/or by insertion of genetic elements, such as transposons, T-DNA, retroviral elements.

Mutations can also be introduced by more specific, targeted introduction of at least one modification by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

The invention further relates to a method of producing a broccoli plant of the invention comprising at least one new trait, the method can comprise introducing a mutation or transgene conferring the at least one new trait into a plant of the invention, wherein a sample of seed of said plant of the invention has been deposited under NCIMB Accession No. 44350. A broccoli plant produced by this method also forms part of the invention.

In still a further embodiment, the invention provides a method of producing a broccoli seed which can comprise crossing a male parent broccoli plant with a female parent broccoli plant and harvesting the resultant broccoli seed, the female parent broccoli plant is a broccoli plant of the invention, e.g. a broccoli plant having all of the morphological or physiological characteristics tabulated herein, including a broccoli plant of broccoli cultivar 25-BO239 RZ, a sample of seed of which having been deposited under NCIMB Accession No. 44350. The resultant broccoli seed produced by this method and the broccoli plant that is produced by growing said broccoli seed also forms part of the invention.

In still a further embodiment, the invention provides a method of producing a broccoli variety which exhibits all of the physiological and morphological characteristics of broccoli variety 25-BO239 RZ, a sample of seed of said variety having been deposited under NCIMB Accession No. 44350.

The invention even further relates to a method of producing a broccoli curd as a food product which can comprise: (a) sowing a seed of a broccoli plant of the invention, (b) growing said seed into a harvestable broccoli plant and (c) harvesting the broccoli curd from the plant. The invention further comprehends packaging and/or processing the broccoli curds.

In a particular embodiment, the method of producing a broccoli curd as a food product which can comprise sowing a seed of variety 25-BO239 RZ, a sample of seed of which having been deposited under NCIMB Accession No. 44350.

In methods of the invention involving producing a broccoli plant or broccoli curd or broccoli head as a food product, the method can involve sowing the seed of the invention in high density as discussed herein, and the sowing can be undertaken optionally mechanically. The harvesting of the broccoli can be by machine, i.e., the harvesting can comprise machine harvesting the broccoli plant or broccoli curd or broccoli head. Each aspect herein discussed of the broccoli of the invention, e.g., that it can be sown in high density, that it has a very short to short height, that the head or curd is about 2 cm to about 10 cm in diameter (such as illustrated in the Figures and described herein, see, e.g., Brief Description of Drawings), that the seed can be mechanically sown, and that the head or curd can be mechanically harvested, and each combination of such aspects, is a surprising and unexpected advancement and distinction over broccoli that heretofore existed.

In one embodiment, the invention relates to a method for mechanically harvesting a broccoli curd, wherein the method comprises the steps of (a) sowing seeds or planting plantlets of broccoli variety 25-BO239 RZ in high density ranging from 20 to 100 plants per m² and (b) cultivating said seeds or plantlets to maturity to obtain a harvestable broccoli plant. The method can include (c) harvesting the curd, advantageously mechanically harvesting the curd, e.g., by a machine that cuts the stem below the curd and above the ground.

In a further embodiment, the invention relates to a method for mechanically harvesting a broccoli curd, wherein the method comprises the steps of (a) sowing seeds or planting plantlets of broccoli variety 25-BO239 RZ in high density ranging from 40 to 80 plants per m² and (b) cultivating said seeds or plantlets to maturity to obtain a harvestable broccoli plant. The method can include (c) harvesting the curd, advantageously mechanically harvesting the curd, e.g., by a machine that cuts the stem below the curd and above the ground.

The invention also relates to a container comprising one or more curds, wherein said curd is harvested from a broccoli plant of the invention, in particular a plant of variety 25-BO239 RZ. Such a container can be a can, a jar, plastic bag, clam shell container (plastic or firm paper or cardboard), cardboard or firm paper or plastic tray that is covered with plastic (such as a cling type of plastic wrap), and the like.

Further encompassed by the invention is a method of determining the genotype of a plant of broccoli plant of the invention, in particular a plant of variety 25-BO239 RZ, a sample of seed of which has been deposited under NCIMB Accession No. 44350, or a first generation progeny thereof, which can comprise obtaining a sample of nucleic acids from said plant and comparing said nucleic acids to a sample of nucleic acids obtained from a reference plant, and detecting a plurality of polymorphisms between the two nucleic acid samples, wherein the plurality of polymorphisms are indicative of broccoli plant of the invention, in particular a plant of variety 25-BO239 RZ and/or give rise to the expression of any one or more, or all, of the physiological and morphological characteristics of the broccoli plant of the invention.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112 (a)), such that Applicants reserve the right and hereby disclose a disclaimer of any previously described product, process of making the product, or method of using the product.

It is noted that in this disclosure and particularly in the claims, terms such as “comprises”, “comprised”, and “comprising” and the like (e.g., “includes”, “included”, “including”, “contains”, “contained”, “containing”, “has”, “had”, “having”, etc.) can have the meaning ascribed to them in U.S. Patent law, i.e., they are open ended terms. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits. Similarly, the terms “consists essentially of” and “consisting essentially of” have the meaning ascribed to them in U.S. Patent law, e.g., they allow for elements not explicitly recited, but exclude elements that are found in the prior art or that affect a basic or novel characteristic of the invention. See also MPEP § 2111.03. In addition, the term “about” is used to indicate that a value includes the standard deviation of error (e.g., “about” indicates that a value can be +/−5% such that “about 1” indicates 0.95 to 1.05).

These and other embodiments are disclosed or are obvious from and encompassed by the following Detailed Description.

### DEPOSITS

The Deposits with NCIMB, Ltd, Wellheads Place, Dyce, Aberdeen AB21 7 GB, UK, on Feb. 12, 2024, under NCIMB accession number 44350 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, can best be understood in conjunction with the accompanying drawing, in which:

FIG. 1 is a photograph comparing on the left side of the photograph broccoli variety Eiffel, whose head is, or wherein the curd is, cut up into sixteen florets, with on the right side of the photograph sixteen individual curds of the present invention broccoli. (Note that as shown in FIG. 1, the head or curd of each plant of the invention provides a small curd, and that an advantage of the present invention is that the curd of the invention is so small it does not need to be further individualized into florets (e.g., the curd of broccoli of the invention is similar in size to a floret of broccoli variety Eiffel). This results in less waste because individualized florets of a regular broccoli curd have yellow edges where they have touched each other and sunlight could not reach where they are touching. These yellowed edges are normally removed before further processing the florets.) At the bottom left side an intact curd harvested of a plant of broccoli variety Eiffel. And at the bottom right two curds harvested of two plants of broccoli variety 25-BO239 RZ, a plant of the invention.

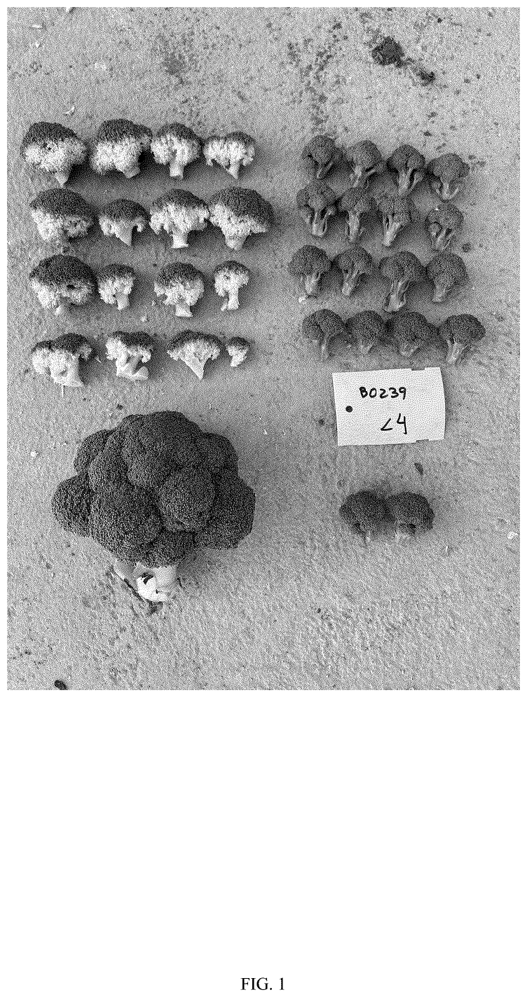

FIG. 2 is a photograph comparing the height of mature plants of a comparative regular broccoli variety (left hand side) with mature plants of the present invention broccoli (right hand side) (note that the comparative broccoli variety Eiffel is about 66 cm in height when sown in high density, whereas present invention broccoli is about 31 cm in height when sown in high density).

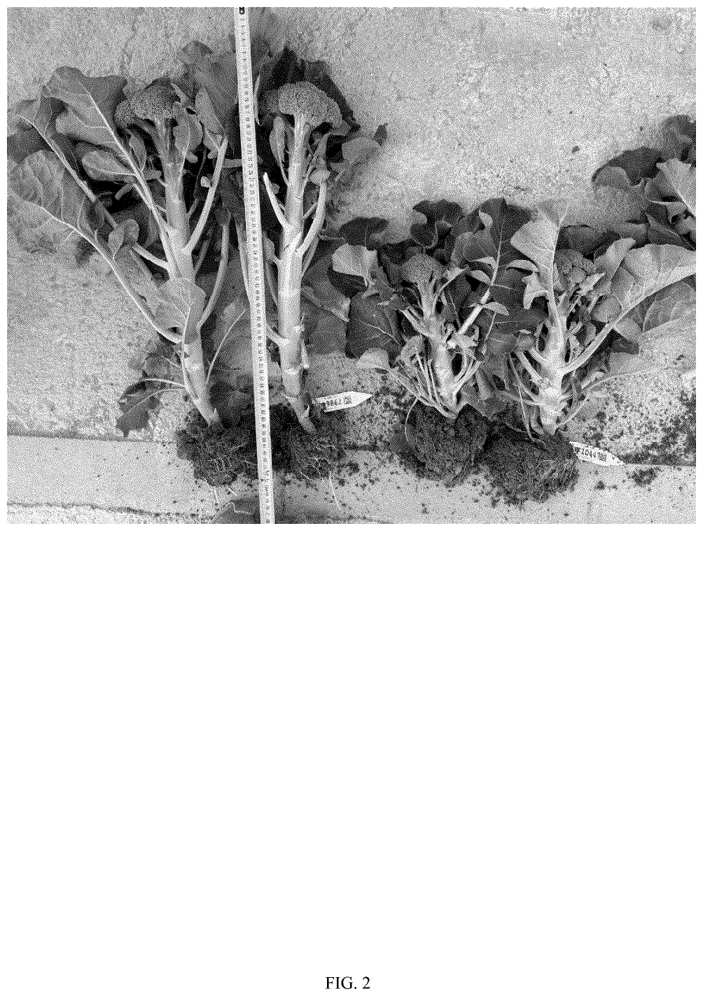

FIG. 3 is a photograph comparing the leaf length and height of mature plants of a comparative regular broccoli variety Eiffel (left hand side) with mature plants of the present invention broccoli 25-BR239 (right hand side).

FIG. 4 is a photograph comparing the leaf length and height of mature plants of a comparative regular broccoli variety Pixel (left hand side) with mature plants of the present invention broccoli 25-BR239 (right hand side).

FIG. 5 is a photograph comparing the leaf length and height of mature plants of a comparative regular broccoli variety Castle Dome (left hand side) with mature plants of the present invention broccoli 25-BR239 (right hand side).

FIG. 6 is a Whisker-plot for comparison trial regarding average leaf length per plant.

FIG. 7 is a Whisker-plot for comparison 1 trial regarding average head diameter.

FIG. 8 is a Whisker-plot for comparison 1 trial regarding average plant height.

### DETAILED DESCRIPTION OF THE INVENTION

The invention provides methods and compositions relating to plants, seeds and derivatives of a new type of broccoli plant and a variety herein referred to as broccoli variety 25-BO239 RZ. Broccoli variety 25-BO239 RZ is a uniform and stable line, distinct from other such lines.

Broccoli variety 25-BO239 RZ was developed by crossing parent lines BO0138 RZ and MSB138 RZ. Line BO0138 RZ was selected in Murcia, Spain, the first cross being made in 2009. The progeny of the first cross was backcrossed with the original line several times and self-pollinated four times to get a stable parent line. Line MSB138 RZ was selected in de Lier, The Netherlands, the first cross being made in 2015. Line MSB138 RZ shows cytoplasmic male sterility and is obtained by crossing BO0138 RZ with MS2333 RZ, wherein MS2333 RZ is the carrier of cytoplasmic male sterility. The progeny of the first cross was backcrossed four times to get a stable parent line. Both parent lines show uniformity and stability. By crossing the parent lines, uniform plants of variety 25-BO239 RZ can be obtained.-In one embodiment, a plant of the invention has all the physiological and morphological characteristics of broccoli variety 25-BO239 RZ. Characteristics of a broccoli plant of the invention, e.g. of variety 25-BO239 RZ, are discussed throughout including in Table 1.

The characteristics as shown in Table 1 are determined according to the UPOV protocol for tests on distinctness uniformity and stability in relation Brassica oleracea L. var. italica Plenck TP/151/2-rev.2. adopted on 21 Apr. 2020 (cpvo.europa.eu/sites/default/files/documents/brassica_oleracea_broccoli_2.2.pdf).

As used herein, a broccoli plant having a very short to short plant height is defined as having a plant height that is in between that of variety New Light, which is a variety showing a very short plant height, and varieties Packman, Chronos, and Primor, which are varieties having a short plant height, when grown under the same conditions. Observations on plant height should be made on fully developed plants just before harvest maturity. To determine plant height, the average height of at least 20 plants in a trial is taken and compared to one or more reference varieties in the same trial in order to determine the correct state of expression (TP/151/2 rev. 2).

In Table 1, the characteristics of “25-BO239 RZ” are shown.

TABLE 1

| Characteristics | |
|---|---|
| Plant height | Very short to short |
| Leaf: number of Lobes | Many |
| Leaf blade: color | Grey green |
| Leaf blade: intensity of color | Medium to dark |
| Head: Level of main head in | Medium |
| relation to plant height | |
| Head: shape in longitudinal | Transverse medium |
| section | elliptic |
| Head: color | Grey green |
| Head: intensity of color | Medium to dark |
| Time of harvest maturity | Early |
| (compared to variety Chronos) | |
| Male sterility | Present |
| Type of cultivation | Annual |

As used herein, head size is determined by measuring the diameter of a head or curd at harvest maturity.

The head size of a plant of the invention, in particular of variety 25-BO239 RZ, is about 2 to about 10 cm, preferably about or greater than 2 cm to less than about 10 cm, when directly sown in high density ranging from 20 to 100 plants per m².

More preferably the head size of a plant of the invention, in particular of variety 25-BO239 RZ, is about or greater than 2 cm to less than about 6 cm, when directly sown in high density ranging from 40 to 80 plants per m².

As used herein, a broccoli plant with leaves having an ‘extra short’ leaf length is defined as a plant having a leaf length that is shorter than that of variety Kechua F1, when grown under the same conditions. Observations on leaf length should be made on fully developed plants just before harvest maturity. To determine leaf length, the average length of all leaves of at least 20 plants in a trial is taken and compared to one or more reference varieties in the same trial in order to determine the correct state of expression. Variety Kechua is a reference variety according the UPOV protocol for tests on distinctness uniformity and stability in relation to Brassica oleracea L. var. italica Plenck (TP/151/2-rev.2. adopted on 21 Apr. 2020), wherein the state of expression for ‘leaf length’ for variety Kechua is ‘short’.

In a particular embodiment the average leaf length of a plant of variety 25-BO239 is less than 30 cm, preferably less than 28 cm, most preferably less than 27.5 cm when sown in high density ranging from 20 to 100 plants per m².

Broccoli variety 25-BO239 RZ when directly sown for summer cultivation takes about 50 to 70 days, preferably about 60 days to reach harvest maturity. In winter cultivation a directly sown plant of broccoli variety 25-BO239 RZ takes about 80 to 100 days to reach harvest maturity.

In an embodiment, the invention relates to broccoli plants that have all the physiological and morphological characteristics of the invention and have acquired said characteristics by introduction of the genetic information that is responsible for the characteristics from a suitable source, either by conventional breeding, or genetic modification, in particular by cisgenesis or transgenesis. Cisgenesis is genetic modification of plants with a natural gene, coding for an (agricultural) trait, from the crop plant itself or from a sexually compatible donor plant. Transgenesis is genetic modification of a plant with a gene from a non-crossable species or a synthetic gene.

Just as useful traits that can be introduced by crossing and selection, useful traits can be introduced directly into the plant of the invention, being a plant of broccoli variety 25-BO239 RZ, by genetic transformation techniques; and, such plants of broccoli variety 25-BO239 RZ that have additional genetic information introduced into the genome or that express additional traits by having the DNA coding there for introduced into the genome via transformation techniques, are within the ambit of the invention, as well as uses of such plants, and the making of such plants.

Genetic transformation can therefore be used to insert a selected transgene into the plant of the invention, being a plant of broccoli variety 25-BO239 RZ or can, alternatively, be used for the preparation of transgenes which can be introduced by backcrossing. Methods for the transformation of plants, including Brassica oleracea, are well known to those of skill in the art.

Vectors used for the transformation of broccoli cells are not limited so long as the vector can express an inserted DNA in the cells. For example, vectors which can comprise promoters for constitutive gene expression in broccoli cells (e.g., cauliflower mosaic virus 35S promoter) and promoters inducible by exogenous stimuli can be used. Examples of suitable vectors include pBI binary vector. The “broccoli cell” into which the vector is to be introduced includes various forms of broccoli cells, such as cultured cell suspensions, protoplasts, leaf sections, and callus. A vector can be introduced into broccoli cells by known methods, such as the polyethylene glycol method, polycation method, electroporation, Agrobacterium-mediated transfer, particle bombardment and direct DNA uptake by protoplasts. To effect transformation by electroporation, one can employ either friable tissues, such as a suspension culture of cells or embryogenic callus or alternatively one can transform immature embryos or other organized tissue directly. In this technique, one would partially degrade the cell walls of the chosen cells by exposing them to pectin-degrading enzymes (pectolyases) or mechanically wound tissues in a controlled manner.

A particularly efficient method for delivering transforming DNA segments to plant cells is microprojectile bombardment. In this method, particles are coated with nucleic acids and delivered into cells by a propelling force. Exemplary particles include those which can be comprised of tungsten, platinum, and preferably, gold. For the bombardment, cells in suspension are concentrated on filters or solid culture medium. Alternatively, immature embryos or other target cells can be arranged on solid culture medium. The cells to be bombarded are positioned at an appropriate distance below the macroprojectile stopping plate. An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target broccoli cells. The screen disperses the particles so that they are not delivered to the recipient cells in large aggregates. It is believed that a screen intervening between the projectile apparatus and the cells to be bombarded reduces the size of projectiles aggregate and can contribute to a higher frequency of transformation by reducing the damage inflicted on the recipient cells by projectiles that are too large. Microprojectile bombardment techniques are widely applicable, and can be used to transform virtually any plant species, including a plant of broccoli variety 25-BO239 RZ. Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations. Moreover, advances in vectors for Agrobacterium-mediated gene transfer have improved the arrangement of genes and restriction sites in the vectors to facilitate the construction of vectors capable of expressing various polypeptide coding genes. The vectors have convenient multi-linker regions flanked by a promoter and a polyadenylation site for direct expression of inserted polypeptide coding genes. Additionally, Agrobacterium containing both armed and disarmed Ti genes can be used for transformation. In those plant strains where Agrobacterium-mediated transformation is efficient, it is the method of choice because of the facile and defined nature of the gene locus transfer. The use of Agrobacterium-mediated plant integrating vectors to introduce DNA into plant cells, including broccoli plant cells, is well known in the art (See, e.g., U.S. Pat. Nos. 7,250,560 and 5,563,055).

Transformation of plant protoplasts also can be achieved using methods based on calcium phosphate precipitation, polyethylene glycol treatment, electroporation, and combinations of these treatments.

A number of promoters have utility for plant gene expression for any gene of interest including but not limited to selectable markers, scoreable markers, genes for pest tolerance, disease resistance, nutritional enhancements and any other gene of agronomic interest. Examples of constitutive promoters useful for broccoli plant gene expression include, but are not limited to, the cauliflower mosaic virus (CaMV) P-35S promoter, a tandemly duplicated version of the CaMV 35S promoter, the enhanced 35S promoter (P-e35S), the nopaline synthase promoter, the octopine synthase promoter, the figwort mosaic virus (P-FMV) promoter (see U.S. Pat. No. 5,378,619), an enhanced version of the FMV promoter (P-eFMV) where the promoter sequence of P-FMV is duplicated in tandem, the cauliflower mosaic virus 19S promoter, a sugarcane bacilliform virus promoter, a commelina yellow mottle virus promoter, the promoter for the thylakoid membrane proteins (psaD, psaF, psaE, PC, FNR, atpC, atpD, cab, rbcS) (see U.S. Pat. No. 7,161,061), the CAB-1 promoter (see U.S. Pat. No. 7,663,027), the promoter from maize prolamin seed storage protein (see U.S. Pat. No. 7,119,255), and other plant DNA virus promoters known to express in plant cells. A variety of plant gene promoters that are regulated in response to environmental, hormonal, chemical, and/or developmental signals can be used for expression of an operably linked gene in plant cells, including promoters regulated by (1) heat, (2) light (e.g., pearbcS-3A promoter, maize rbcS promoter, or chlorophyll a/b-binding protein promoter), (3) hormones, such as abscisic acid, (4) wounding (e.g., wunl, or (5) chemicals such as methyl jasmonate, salicylic acid, or Safener. It can also be advantageous to employ organ-specific promoters.

Exemplary nucleic acids which can be introduced to the broccoli variety of this invention include, for example, DNA sequences or genes from another species, or even genes or sequences which originate with or are present in broccoli species, but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene can already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Many hundreds if not thousands of different genes are known and could potentially be introduced into a plant of broccoli variety 25-BO239 RZ. Non-limiting examples of particular genes and corresponding phenotypes one can choose to introduce into a broccoli plant include one or more genes for insect tolerance, pest tolerance such as genes for fungal disease control, herbicide tolerance, and genes for quality improvements such as yield, nutritional enhancements, environmental or stress tolerances, or any desirable changes in plant physiology, growth, development, morphology or plant product(s).

Alternatively, the DNA coding sequences can affect these phenotypes by encoding a non-translatable RNA molecule that causes the targeted inhibition of expression of an endogenous gene, for example via antisense-or cosuppression-mediated mechanisms. The RNA could also be a catalytic RNA molecule (i.e., a ribozyme) engineered to cleave a desired endogenous mRNA product. Thus, any gene which produces a protein or mRNA which expresses a phenotype or morphology change of interest is useful for the practice of the present invention. (See also U.S. Pat. No. 7,576,262, “Modified gene-silencing RNA and uses thereof.”)

U.S. Pat. Nos. 7,230,158, 7,122,720, 7,081,363, 6,734,341, 6,503,732, 6,392,121, 6,087,560, 5,981,181, 5,977,060, 5,608,146, 5,516,667, each of which, and all documents cited therein are hereby incorporated herein by reference, consistent with the above INCORPORATION BY REFERENCE section, are additionally cited as examples of U.S. Patents that can concern transformed broccoli and/or methods of transforming broccoli or broccoli plant cells, and techniques from these U.S. Patents, as well as promoters, vectors, etc., can be employed in the practice of this invention to introduce exogenous nucleic acid sequence(s) into a plant of broccoli variety 25-BO239 RZ (or cells thereof), and exemplify some exogenous nucleic acid sequence(s) which can be introduced into a plant of broccoli variety 25-BO239 RZ (or cells thereof) of the invention, as well as techniques, promoters, vectors etc., to thereby obtain further plants of broccoli variety 25-BO239 RZ, plant parts and cells, seeds, other propagation material harvestable parts of these plants, etc. of the invention, e.g. tissue culture, including a cell or protoplast, such as an embryo, meristem, cotyledon, pollen, leaf, anther, root, root tip, pistil, flower, seed or stalk.

The invention further relates to propagation material for producing plants of the invention. Such propagation material can comprise inter alia seeds of the claimed plant and parts of the plant that are involved in sexual reproduction. Such parts are for example selected from the group consisting of seeds, microspores, ovaries, ovules, embryo sacs and egg cells. In addition, the invention relates to propagation material which may comprise parts of the plant that are suitable for vegetative reproduction, for example cuttings, roots, stems, cells, protoplasts.

According to a further aspect thereof the propagation material of the invention can comprise a tissue culture of the claimed plant. The tissue culture can comprise regenerable cells. Such tissue culture can be derived from leaves, pollen, embryos, cotyledon, hypocotyls, meristematic cells, roots, root tips, flowers, seeds and stems. (See generally U.S. Pat. No. 7,041,876 on broccoli being recognized as a plant that can be regenerated from cultured cells or tissue).

Also, the invention comprehends methods for producing a seed of a “25-BO239 RZ”-derived broccoli plant which can comprise (a) crossing a plant of broccoli variety 25-BO239 RZ, a sample of seed of which having been deposited under NCIMB Accession No. 44350, with a second broccoli plant, and (b) whereby seed of a 25-BO239 RZ-derived broccoli plant form. Such a method can further comprise (c) crossing a plant grown from 25-BO239 RZ-derived broccoli seed with a second broccoli plant to yield additional 25-BO239 RZ-derived broccoli seed, (d) growing the additional 25-BO239 RZ-derived broccoli seed of step (c) to yield additional 25-BO239 RZ-derived broccoli plants, and (e) repeating the crossing and growing of steps (c) and (d) for an additional 3-10 generations to further generate 25-BO239 RZ-derived broccoli plants.

The invention further relates to the above methods that can further comprise selecting at steps b), d), and e), a 25-BO239 RZ-derived broccoli plant, exhibiting one or more or all of the physiological and morphological characteristics of broccoli variety 25-BO239 RZ, a sample of seed of said variety having been deposited under NCIMB Accession No. 44350, and other selected traits.

The invention further involves a method of determining the genotype of a plant of broccoli variety 25-BO239 RZ, a sample of seed of which has been deposited under NCIMB Accession No. 44350, or a first generation progeny thereof, which can comprise obtaining a sample of nucleic acids from said plant and detecting in said nucleic acids a plurality of polymorphisms. This method can additionally comprise the step of storing the results of detecting the plurality of polymorphisms on a computer readable medium. The plurality of polymorphisms are indicative of and/or give rise to the expression of the physiological and morphological characteristics of broccoli variety 25-BO239 RZ.

There are various ways of obtaining genotype data from a nucleic acid sample. Genotype data can be gathered which is specific for certain phenotypic traits (e.g. gene sequences), but also patterns of random genetic variation can be obtained to construct a so-called DNA fingerprint. Depending on the technique used a fingerprint can be obtained that is unique for broccoli variety 25-BO239 RZ. Obtaining a unique DNA fingerprint depends on the genetic variation present in a variety and the sensitivity of the fingerprinting technique. A technique known in the art to provide a good fingerprint profile is called AFLP fingerprinting technique (See generally U.S. Pat. No. 5,874,215), but there are many other marker based techniques, such as RFLP (or Restriction fragment length polymorphism), SSLP (or Simple sequence length polymorphism), RAPD (or Random amplification of polymorphic DNA) VNTR (or Variable number tandem repeat), Microsatellite polymorphism, SSR (or Simple sequence repeat), STR (or Short tandem repeat), SFP (or Single feature polymorphism) DArT (or Diversity Arrays Technology), RAD markers (or Restriction site associated DNA markers) (e.g. Baird et al. PloS One Vol. 3 e3376, 2008; Semagn et al. African Journal of Biotechnology Vol. 5 number 25 pp. 2540-2568, 29 Dec., 2006). Nowadays, sequence-based methods are utilizing Single Nucleotide Polymorphisms (SNPs) that are randomly distributed across genomes, as a common tool for genotyping (e.g. Elshire et al. PloS One Vol. 6: e19379, 2011; Poland et al. PloS One Vol. 7: e32253; Truong et al. PLOS One Vol. 7 number 5: e37565, 2012).

With any of the aforementioned genotyping techniques, polymorphisms can be detected when the genotype and/or sequence of the plant of interest is compared to the genotype and/or sequence of one or more reference plants. As used herein, the genotype and/or sequence of a reference plant can be derived from, but is not limited to, any one of the following: parental lines, closely related plant varieties or species, complete genome sequence of a related plant variety or species, or the de novo assembled genome sequence of one or more related plant varieties or species.

The polymorphism revealed by these techniques can be used to establish links between genotype and phenotype. The polymorphisms can thus be used to predict or identify certain phenotypic characteristics, individuals, or even species. The polymorphisms are generally called markers. It is common practice for the skilled artisan to apply molecular DNA techniques for generating polymorphisms and creating markers.

The polymorphisms of this invention can be provided in a variety of mediums to facilitate use, e.g. a database or computer readable medium, which can also contain descriptive annotations in a form that allows a skilled artisan to examine or query the polymorphisms and obtain useful information.

As used herein “database” refers to any representation of retrievable collected data including computer files such as text files, database files, spreadsheet files and image files, printed tabulations and graphical representations and combinations of digital and image data collections. In a preferred aspect of the invention, “database” refers to a memory system that can store computer searchable information.

As used herein, “computer readable media” refers to any medium that can be read and accessed directly by a computer. Such media include, but are not limited to: magnetic storage media, such as floppy discs, hard disc, storage medium and magnetic tape; optical storage media such as CD-ROM; electrical storage media such as RAM, DRAM, SRAM, SDRAM, ROM; and PROMs (EPROM, EEPROM, Flash EPROM), and hybrids of these categories such as magnetic/optical storage media. A skilled artisan can readily appreciate how any of the presently known computer readable mediums can be used to create a manufacture which can comprise computer readable medium having recorded thereon a polymorphism of the present invention.

As used herein, “recorded” refers to the result of a process for storing information in a retrievable database or computer readable medium. For instance, a skilled artisan can readily adopt any of the presently known methods for recording information on computer readable medium to generate media which can comprise the polymorphisms of the present invention. A variety of data storage structures are available to a skilled artisan for creating a computer readable medium where the choice of the data storage structure will generally be based on the means chosen to access the stored information. In addition, a variety of data processor programs and formats can be used to store the polymorphisms of the present invention on computer readable medium.

The present invention further provides systems, particularly computer-based systems, which contain the polymorphisms described herein. Such systems are designed to identify the polymorphisms of this invention. As used herein, “a computer-based system” refers to the hardware, software and memory used to analyze the polymorphisms. A skilled artisan can readily appreciate that any one of the currently available computer-based system are suitable for use in the present invention.

Broccoli and broccoli florets are sold in packaged form. For instance, broccoli heads or curds are sold in a wrap or bag, such as plastic wrap or bag. Broccoli florets are sold in various forms, including in a bag or wrap, such as a plastic bag or wrap, as well as in cans, jars, clam shell containers, firm trays (e.g., plastic, Styrofoam, cardboard, firm paper) that are covered with an overwrap, such as a plastic overwrap, and boxes (such as cardboard boxes that may have wax coating and/or a paper overwrap labeling; for instance, in the case of frozen broccoli). In some instances, there is a modified atmosphere in the packaging to help extend shelf life. As shown by the Figures herewith and the Brief Description of the Drawings, a curd of broccoli of the invention can be similar in size to a floret of other broccoli varieties, such as Eiffel. Accordingly, the curd of the invention can be harvested, e.g., mechanically harvested, and sold in a packaged form that is currently used for broccoli florets of other varieties, such as Eiffel. Thus, a plurality of curds of broccoli of the invention can be in a package that would currently contain a plurality of florets of another broccoli variety, such as Eiffel. Also, of course, singular curds of the invention can be packaged in the same fashion as singular curds of other varieties.

The invention is further described by the following numbered paragraphs:
1. A broccoli (Brassica oleracea var. italica) seed capable of growing into a broccoli plant having a set of characteristics including but not limited to a short to very short plant height, and a head size of about 2 to about 10 cm when directly sown in high density ranging from 20 to 100 plants per m², representative seed comprising genetic information that confers said characteristics having been deposited under NCIMB Accession No. 44350.
2. The seed of paragraph 1, wherein the plant grown from said seed further comprises the characteristic of having extra short leaves.
3. The seed of paragraph 1, wherein the seed belongs to a broccoli variety designated 25-BO239 RZ, a sample of seed of said variety having been deposited under NCIMB Accession No. 44350.
4. A broccoli plant grown from the seed of paragraph 1.
5. A broccoli plant grown from the seed of paragraph 2.
6. A broccoli plant grown from the seed of paragraph 3.
7. The broccoli plant described in paragraph 4 or 5, or a part thereof, having all the physiological and morphological characteristics of the broccoli plant grown from seed deposited under NCIMB Accession No. 44350 or of broccoli variety designated 25-BO239 RZ.
8. A part of the broccoli plant described in any one of paragraphs 4-7, wherein said part comprises a microspore, ovary, ovule, embryo sac, egg cell, cutting, root, stem, cell or protoplast.
9. A tissue culture of regenerable cells or protoplasts from the broccoli plant of paragraph 4 or 5.
10. The tissue culture of paragraph 9, wherein said cells or protoplasts of the tissue culture are derived from a tissue comprising a leaf, embryo, cotyledon, hypocotyl, meristematic cell, root, root tip, flower, seed or stem.
11. A broccoli plant regenerated from the tissue culture of paragraph 9 or 10, wherein the regenerated plant expresses all of the physiological and morphological characteristics of broccoli variety 25-BO239 RZ, a sample of seed of said variety having been deposited under NCIMB Accession No. 44350.
12. A method of vegetatively propagating a plant of broccoli variety 25-BO239 RZ comprising (a) collecting tissue capable of being propagated from a broccoli plant as described in any one of paragraphs 4-7, (b) cultivating the tissue to obtain proliferated shoots and rooting the proliferated shoots to obtain rooted plantlets, and (c) optionally growing plants from the rooted plantlets.
13. A method for producing a progeny plant of broccoli variety 25-BO239 RZ, comprising crossing the broccoli plant as described in any of paragraphs 4-7 with another broccoli plant, harvesting the resultant seed, and growing said seed.
14. A progeny plant produced by the method of paragraph 13, wherein said progeny exhibits all the morphological and physiological characteristics of the broccoli variety designated 25-BO239 RZ, a sample of seed of said variety having been deposited under NCIMB Accession No. 44350.
15. The broccoli seed as described in any of paragraphs 1-3, or the broccoli plant as described in any one of paragraphs 4-7 and 11, or the broccoli plant from the method of paragraph 13 further comprising a transgene.
16. The seed or plant of paragraph 15, wherein the transgene is introduced via transformation.
17. A method for producing a modified broccoli plant comprising mutagenizing the seed of any one of paragraphs 1-3 and growing said seed.
18. A method for producing a modified broccoli plant comprising mutagenizing the broccoli plant of any one of paragraphs 4-7 and 11 or the broccoli plant produced by the method of paragraph 13.
19. A method for producing a modified broccoli plant comprising mutagenizing the plant part of paragraph 8.
20. A method for producing a modified broccoli plant comprising mutagenizing the tissue culture of paragraph 9 or 10.
21. A method of producing a broccoli seed comprising crossing a male parent broccoli plant with a female parent broccoli plant and harvesting the resultant broccoli seed, wherein said female parent broccoli plant is the broccoli plant as described in any one of paragraphs 4-7 and 11 or the broccoli plant produced by the method of paragraph 13.
22. An F1 broccoli seed produced by the method of paragraph 21.
23. A broccoli plant produced by growing the seed of paragraph 22.
24. A method for producing a seed of a 25-BO239 RZ-derived broccoli plant comprising (a) crossing a plant of broccoli variety 25-BO239 RZ, a sample of seed of which having been deposited under NCIMB Accession No. 44350, with a second broccoli plant, and (b) whereby seed of a 25-BO239 RZ-derived broccoli plant forms.
25. A method for producing a broccoli curd as a food product comprising sowing the seed of any one of paragraphs 1-3 and growing the seed into a harvestable broccoli plant and harvesting the curd of said plant, optionally processing and/or packaging the curd; or growing the broccoli plant as described in any one of paragraphs 4-7 and 11 or the broccoli plant produced by the method of paragraph 13, into a harvestable broccoli plant and harvesting the curd of said plant, optionally processing and/or packaging the curd.
26. A method for mechanically harvesting a broccoli curd, comprising the steps of (a) sowing seeds or planting plantlets of broccoli variety 25-BO239 RZ in high density ranging from 20 to 100 plants per m², (b) cultivating said seeds or plantlets to maturity to obtain a harvestable broccoli plant, and (c) harvesting the curd of said broccoli plant.
27. The method of paragraph 26, wherein step (a) comprises sowing seeds or planting plantlets of broccoli variety 25-BO239 RZ in high density ranging from 40 to 80 plants per m².
28. A container comprising one or more broccoli curds, wherein the one or more broccoli curds are harvested from the plant as described in any one of paragraphs 4-7 and 11 or the plant produced by the method of paragraph 13; or wherein the one or more broccoli curds are harvested in the method of paragraph 25.

### Examples

### Comparison Trial

Plants of the of the invention (Variety 25-BO239) and hybrid varieties Chronos, Eiffel, Pixel, and Castle Dome were sown on 11 Sep. 2024 and transplanted to a field near Cartagena (Spain) on 10 Oct. 2024. From each variety 27 to 30 plants were evaluated. Nine weeks after transplanting the plants to the field, they were evaluated for the following characteristics: plant height (from ground to top of curd), curd diameter, and leaf length.

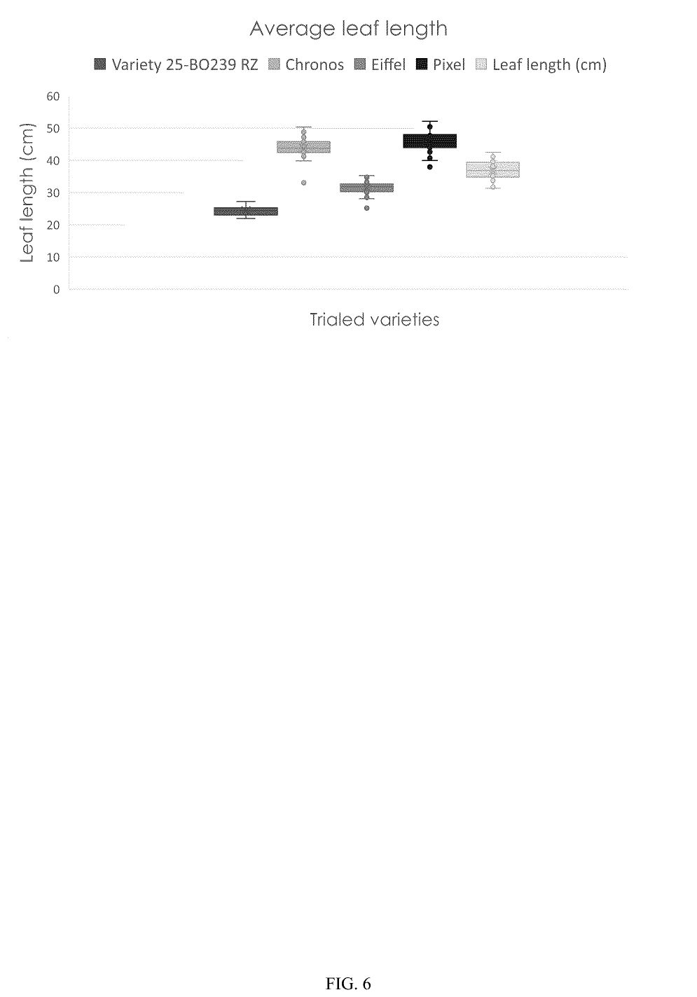

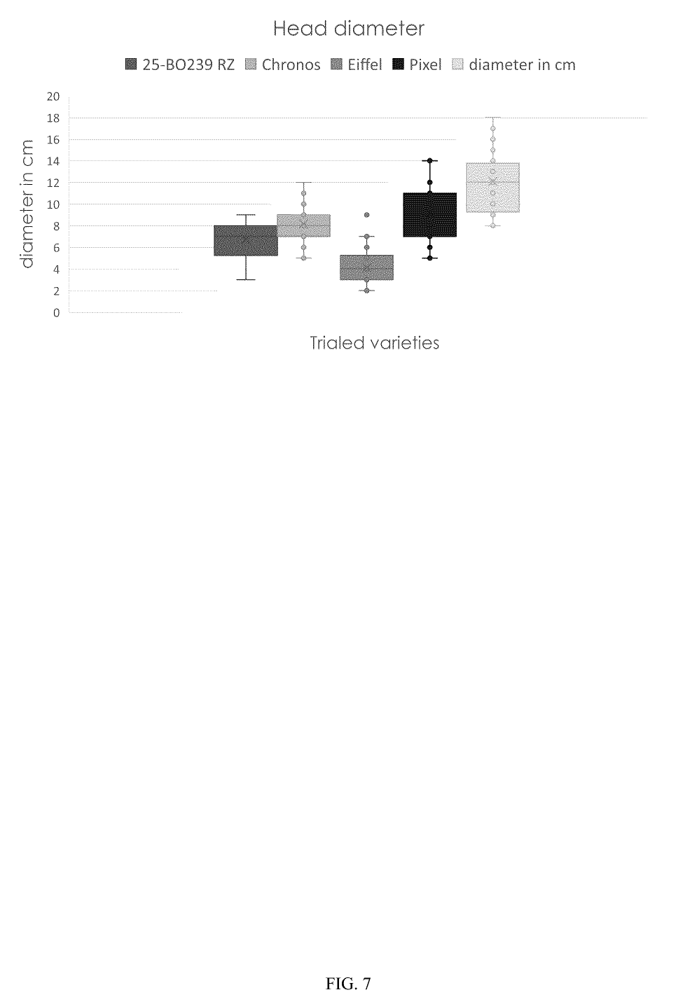

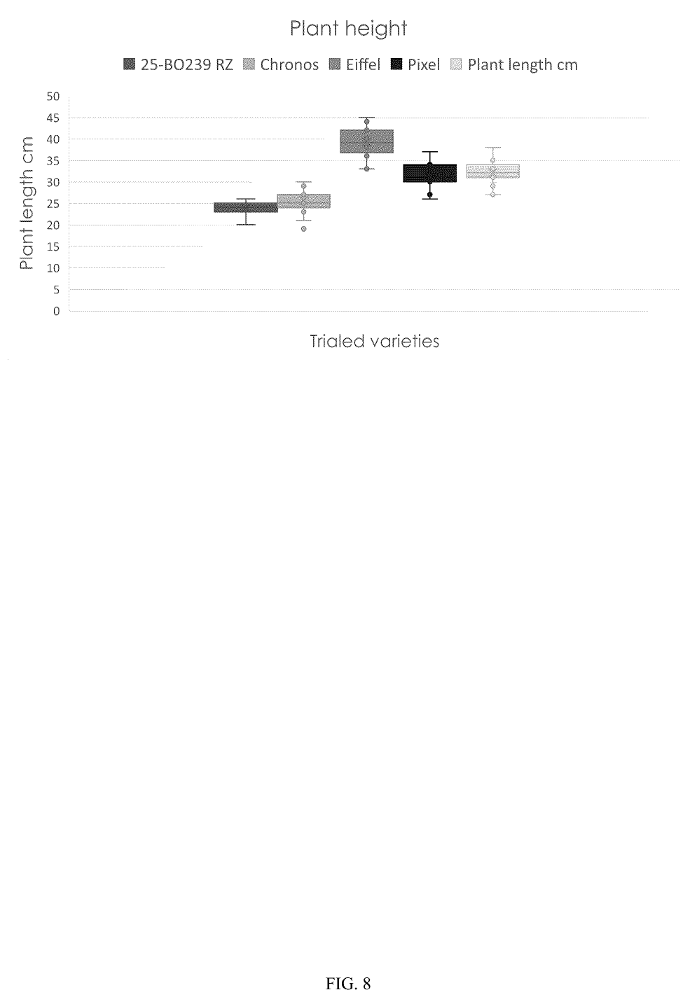

Leaf length was measured from where it is attached to the stem, to the tip of the leaf blade. For each plant all leaves were measured. The average of each plant is summarized in the Whisker-plot of FIG. 6. The measurements for head diameter and plant height of each plant are also visualized in a Whisker plot (FIG. 7 and FIG. 8 respectively).

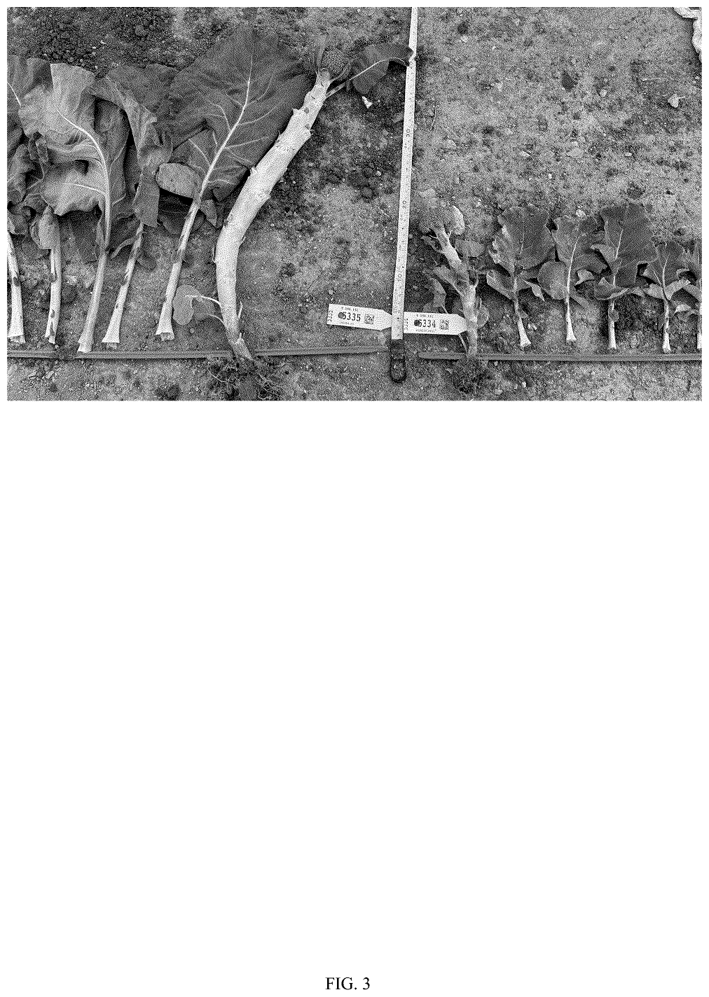

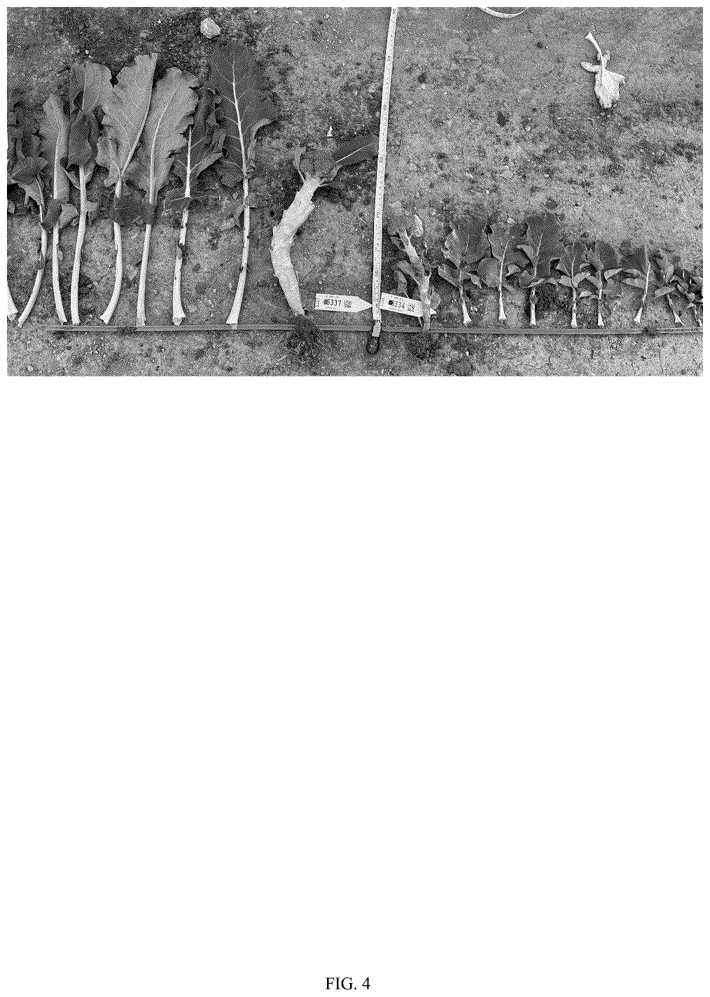

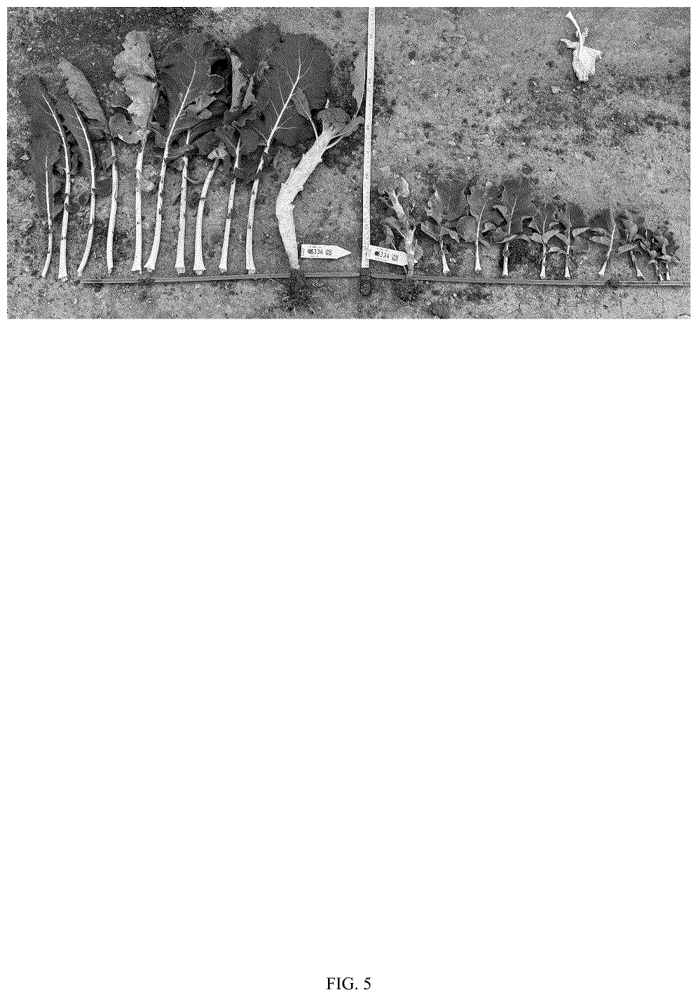

From the results it is clear that the average leaf length for plants of the invention is significantly different compared to that of the other varieties Chronos, Eiffel, Pixel, and Castle Dome (FIG. 6). This is further illustrated by FIG. 3-5 where the plant of the invention and leaves separated thereof (right hand side) are compared to plants of and leaves separated thereof of varieties Eiffel, Pixel and Castle Dome (all left hand side), respectively.

Furthermore, as can be seen in FIG. 8, plants of the invention have a similar plant height as plants of variety Chronos. However, Chronos has much larger leaves making it less suitable for mechanical harvesting.

Looking at the data overall, the variety of the invention shows to have a more uniform appearance compared to the other varieties when sown in high density, since it has far less outliers for the characteristics that have been measured.

Having thus described in detail preferred embodiments of the present invention, it is to be understood that the invention is not to be limited to particular details set forth in the above description as many apparent variations thereof are possible without departing from the spirit or scope of the present invention.

What is claimed is:

1. A broccoli (Brassica oleracea var. italica) seed capable of growing into a broccoli plant having a set of characteristics including but not limited to a short to very short plant height, and a head size of about 2 to about 10 cm when directly sown in high density ranging from 20 to 100 plants per m², representative seed comprising genetic information that confers said characteristics having been deposited under NCIMB Accession No. 44350.

2. The seed of claim 1, wherein the plant grown from said seed further comprises the characteristic of having extra short leaves.

3. The seed of claim 1, wherein the seed belongs to a broccoli variety designated 25-BO239 RZ, a sample of seed of said variety having been deposited under NCIMB Accession No. 44350.

4. A broccoli plant grown from the seed of claim 1.

5. A broccoli plant grown from the seed of claim 2

6. A broccoli plant grown from the seed of claim 3.

7. The broccoli plant of claim 4, or a part thereof, having all the physiological and morphological characteristics of the broccoli plant grown from seed deposited under NCIMB Accession No. 44350 or of broccoli variety designated 25-BO239 RZ.

8. A part of the broccoli plant of any one of claims 4-7, wherein said part comprises a microspore, ovary, ovule, embryo sac, egg cell, cutting, root, stem, cell or protoplast.

9. A tissue culture of regenerable cells or protoplasts from the broccoli plant of claim 4 or 5.

10. The tissue culture of claim 9, wherein said cells or protoplasts of the tissue culture are derived from a tissue comprising a leaf, embryo, cotyledon, hypocotyl, meristematic cell, root, root tip, flower, seed or stem.

11. A broccoli plant regenerated from the tissue culture of claim 9, wherein the regenerated plant expresses all of the physiological and morphological characteristics of broccoli variety 25-BO239 RZ, a sample of seed of said variety having been deposited under NCIMB Accession No. 44350.

12. A method of vegetatively propagating a plant of broccoli variety 25-BO239 RZ comprising (a) collecting tissue capable of being propagated from the broccoli plant of claim 4, (b) cultivating the tissue to obtain proliferated shoots and rooting the proliferated shoots to obtain rooted plantlets, and (c) optionally growing plants from the rooted plantlets.

13. A method for producing a progeny plant of broccoli variety 25-BO239 RZ, comprising crossing the broccoli plant of claim 4 with another broccoli plant, harvesting the resultant seed, and growing said seed.

14. A progeny plant produced by the method of claim 13, wherein said progeny exhibits all the morphological and physiological characteristics of the broccoli variety designated 25-BO239 RZ, a sample of seed of said variety having been deposited under NCIMB Accession No. 44350.

15. The broccoli seed of any one of claims 1-3, or the broccoli plant of claim 4, further comprising a transgene.

16. The seed or the plant of claim 15, wherein the transgene is introduced via transformation.

17. A method for producing a modified broccoli plant comprising mutagenizing the seed of claim 1 and growing said seed.

18. A method for producing a modified broccoli plant comprising mutagenizing the broccoli plant of claim 4 or the broccoli plant produced by the method of claim 13.

19. A method for producing a modified broccoli plant comprising mutagenizing the plant part of claim 8.

20. A method for producing a modified broccoli plant comprising mutagenizing the tissue culture of claim 9.

21. A method of producing a broccoli seed comprising crossing a male parent broccoli plant with a female parent broccoli plant and harvesting the resultant broccoli seed, wherein said female parent broccoli plant is the broccoli plant of claim 4.

22. An F1 broccoli seed produced by the method of claim 21.

23. A broccoli plant produced by growing the seed of claim 22.

24. A method for producing a seed of a 25-BO239 RZ-derived broccoli plant comprising (a) crossing a plant of broccoli variety 25-BO239 RZ, a sample of seed of which having been deposited under NCIMB Accession No. 44350, with a second broccoli plant, and (b) whereby seed of a 25-BO239 RZ-derived broccoli plant forms.

25. A method for producing a broccoli curd as a food product comprising sowing the seed of any one of claims 1-3 and growing the seed into a harvestable broccoli plant and harvesting the curd of said plant, optionally processing and/or packaging the curd; or growing the broccoli plant of claim 4, into a harvestable broccoli plant and harvesting the curd of said plant, optionally processing and/or packaging the curd.

26. A method for mechanically harvesting a broccoli curd, comprising the steps of (a) sowing seeds or planting plantlets of broccoli variety 25-BO239 RZ in high density ranging from 20 to 100 plants per m², (b) cultivating said seeds or plantlets to maturity to obtain a harvestable broccoli plant, and (c) harvesting the curd of said broccoli plant.

27. The method of claim 26, wherein step (a) comprises sowing seeds or planting plantlets of broccoli variety 25-BO239 RZ in high density ranging from 40 to 80 plants per m².

28. A container comprising one or more broccoli curds, wherein the one or more broccoli curds are harvested from the plant of claim 4.
